(19) United States
(12) Patent Application Publication
(10) Pub. No.: US 2012/0150213 A1
(43) Pub. Date: Jun. 14, 2012

(54) MEDICAL COMPRESSION DEVICE

(76) Inventor: Charles Samuel Squire Gordon, St. Pete Beach, FL (US)

(21) Appl. No.: 13/315,420

(22) Filed: Dec. 9, 2011

Related U.S. Application Data
(60) Provisional application No. 61/421,857, filed on Dec. 10, 2010.

(51) Int. Cl. A61B 17/12

(52) U.S. Cl. 606/201

(57) ABSTRACT

A compression apparatus having a tension device configured to be secured relative to a work area. A compression member is adjustably coupled to the tension device. The compression member having a contact surface configured to apply pressure relative to an anatomical location.

### CROSS-REFERENCE TO RELATED APPLICATIONS

This application claims priority to U.S. Provisional Patent Application No. 61/421,857, filed Dec. 10, 2010 the entire disclosure of which is incorporated herein by reference.

### TECHNICAL FIELD

This disclosure relates to medical devices and, more particularly, to medical compression devices providing compression to an anatomical location.

### BACKGROUND

Many interventional procedures, such as cardiac cauterizations, diagnostic arteriograms, arterial based interventions such as carotid stenting, and newer interventions such as thoracic and abdominal aortic aneurysm repair, utilize access through the common femoral artery. The common femoral artery is frequently entered at a point which is directly over the femoral head. The femoral head may provide a solid base so that pressure applied to the area after the intervention adequately compresses the artery to stop the bleeding at the site. If a physician accesses the common femoral artery directly from the skin into the artery at a 90 degree angle then the access sheath can become kinked and injury to the artery can occur not to mention the inability to complete the intervention. Thus, a more favorable angle of entry into the vessel may often be closer to 45 degrees, which usually means that the entry point into the skin may often be well below the entry site into the artery for retrograde (toward the head) punctures. It is not unusual that pressure applied over the skin at the access sheath entry site, does not adequately compress the artery at the arterial entry point which can cause complications such as groin hematomas, pseudoaneurysms of the common femoral artery requiring surgical intervention, to life threatening retroperitoneal hemorrhages.

### SUMMARY OF DISCLOSURE

According to a first embodiment, a compression apparatus includes a tension device that may be configured to be secured relative to a work area. The compression apparatus may further include a compression member adjustably coupled to the tension device. The compression member may include a contact surface configured to apply pressure relative to an anatomical location.

One or more of the following features may be included. The compression member may include a plurality of prongs. The compression member may include at least two prongs. The compression member may include at least a ring. The compression member may include at least two outer prongs and two inner prongs. At least a portion of the plurality of prongs may be telescopically adjustable. The at least two prongs may be staggered. The at least two prongs may be on a same plane. The compression member may further include an articulable joint feature proximal the tension device. The tension device may include a fixture that may have at least one articulable joint that may be pivotally attached to a plurality of sections.

According to another embodiment, a compression apparatus may include a tension device secured relative to a work area. A compression member may be adjustably coupled to the tension device, and may include a contact surface configured to apply pressure relative to an anatomical location. The compression apparatus may further include at least one articulable joint attached between the compression member and the tension device.

The compression member may include a plurality of prongs. The compression member may include a least a ring. The at least one articulable joint may be at least partially disposed between at least two prongs. The at least a ring may be individually adjustable. The tension device may include a Bookwalter retractor. The at least two prongs may be staggered. The at least two prongs may be on a same plane. The at least two prongs may be individually adjustable. The at least two prongs may include two outer prongs and two inner prongs. The two outer prongs may be stationary and the two inner prongs may be individually adjustable. The inner prongs may be telescopically adjustable.

According to yet another embodiment, a compression apparatus includes a connection feature, at least one contact member associated with the connection feature and an attachment device associated with the connection feature for positioning of the at least one contact member relative to an anatomical work area.

The connection feature may be a crosspiece. The contact member may include at least two prongs. The contact member may be at least a ring. The at least two prongs may be fixedly attached to the connection feature. The at least two prongs may be individually adjustable. The at least two prongs may include two outer prongs and two inner prongs. The two outer prongs may be stationary and the two inner prongs may be individually adjustable.

The details of one or more implementations are set forth in the accompanying drawings and the description below. Other features and advantages will become apparent from the description, the drawings, and the claims.

### BRIEF DESCRIPTION OF THE DRAWINGS

FIG. 1 depicts an embodiment of a compression apparatus having a tension device and a compression member.

FIG. 1A depicts an embodiment a contact member.

FIG. 2 depicts an embodiment of a hydraulic system.

FIG. 3 depicts an embodiment of a prong in the form of a ring.

FIG. 4 depicts a cross-sectional view of a ring over an anatomical location.

FIG. 5 depicts an embodiment of the compression member having adjustable prongs.

FIG. 6 depicts another embodiment of the compression member having adjustable prongs that rotate around an axis.

FIG. 7A depicts perspective view of a compression member with staggered configuration.

FIG. 7B depicts cross-sectional view of a compression member with staggered configuration.

FIG. 8 depicts an embodiment of the compression member having telescoping prongs.

Like reference symbols in the various drawings indicate like elements.

### DETAILED DESCRIPTION OF THE EMBODIMENTS

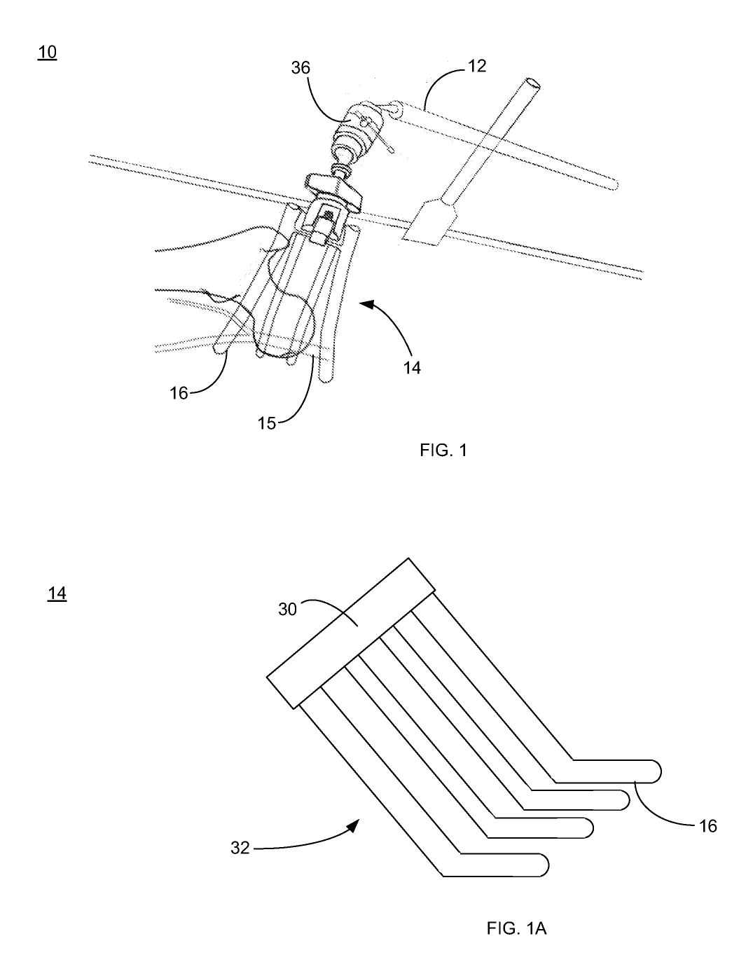

Referring to FIGS. 1 and 1A, an embodiment of compression apparatus 10 may generally include tension device 12 configured to be secured relative to a work area. Compression apparatus 10 may further include a compression member 14 adjustably coupled to tension device 12. Compression member 14 may include contact surface 16 which may be configured to apply pressure relative to an anatomical location.

Compression apparatus 10 may be used anywhere there is a need to, for example access the common femoral artery or other vascular aspect (i.e. artery 15). For example, accessing the common femoral artery in procedures such as heart cauterizations, cerebral angiograms, carotid stenting, thoracic and abdominal aortic artery repairs and upper and lower extremity arteriograms with or without interventions. Compression apparatus 10 may, for example, provide for femoral artery compression in a precisely controlled, sterile manner that can be deployed before, during, or after puncture of the femoral artery for diagnostic and/or arterial based interventions. While the description herein may generally describe the application of compression apparatus 10 in the context of surgical aid for use during various femoral interventional procedures, such description is intended for the purpose of illustration, and not of limitation. Compression apparatus 10 may suitably be utilized in connection with other vascular access procedures and/or other medical procedures, including vascular access or otherwise. Artery 15 is diagrammatically depicted for the purpose of illustration and explanation, and may not be exposed and/or visible as depicted during use of the compression apparatus 10.

Compression member 14 may be utilized to provide the actual contact with an anatomical location while performing any of the above procedures. Compression member 14 may be utilized as a surgical assist device that may allow for controlled pressure application to the anatomical location. For example, compression member 14 may be used in an angiography suite, a cardiology suite and an operating room. For example, compression member 14 may be used to compress an anatomical location such as compressing an artery (e.g., by applying a compressive pressure to a patient's skin overlying the artery) above and below a vascular sheath.

In some embodiments, compression member 14 may be adjusted in a manner to allow for precise pressure when applying compression to an anatomical location, e.g., which may be associated with an arterial intervention. For example, increased force may be applied via compression member 14 after the arterial intervention to adequately compress the artery to assist in stopping the bleeding at the anatomical site that may include the intervention access site. In some embodiments, the pressure applied by compression member 14 may be decreased gradually as the body's own clotting mechanism seals the intervention access site (e.g., which may include a puncture and/or incision).

Compression member 14 may be formed from various materials. For example, in some embodiments, compression member 14 may be made of a plastic material and thus disposable, may be made of a carbon fiber material and thus be radiolucent, and/or may be made of a metallic material (e.g., such as aluminum, stainless steel, titanium, or the like) and thus may be durable and sterilized for repeated use, e.g., in more than on procedure. It is understood that the above are only examples of compression member 14 and should not be construed as limitations, as compression member 14 may be constructed for other materials.

Compression member 14 may be attached to tension device 12. Tension device 12 may maintain a relative position of compression member 14, and thereby enable compression member 14 to provide the desired pressure during use in any of the aforementioned procedures. Tension device 12 may be attached to a work area, and/or a feature associated with the work area, to provide stable support when applying pressure through compression member 14. For example, tension device 12 may be attached (e.g., removably and/or permanently) to a surgical work surface and/or a support structure of a surgical work surface. Tension device 12 may similarly be attached to various other stationary and/or movable features associated with the work area.

For example, compression member 14 may include one or more contact members 32. In an example embodiment depicted in FIG. 1A, the one or more contact members 32 may include one or more prongs.

In an example embodiment in which the contact feature may include a plurality of prongs, connection feature 30 may include a crosspiece. Additionally, connection feature 30 may include a joint feature or a point where all the prongs of contact member 32 meet. The crosspiece may include a plate or a rod. The plate may be configured to be at different angles relative to compression member 14. The joint feature may be a weld or solder point that connects all the prongs together. Some of the prongs may be made from one continuous piece that curves at the top of contact member 32 to create a point where it may be possible to attach to tension device 12 through an attachment device 36.

Contact member 32 may include at least two prongs. Contact member 32 may be a collection of prongs or one or more rings attached to connection feature 30. In some embodiments, compression member 14 may include contact member 32 and connection feature 30. Contact member 32 may include a number of prongs or rings, which may also include contact surfaces 16.

Referring to FIG. 1, attachment device 36 may be fixed, adjustable, and/or may include a joint. Attachment device 36 may allow an operator to position compression device 14 relative to the anatomical location. Attachment device 36 may be secured in place (e.g., in a desired orientation and/or position) using a locking mechanism that might comprise threading, latching or clamping. In some embodiments, attachment device 36 may be integrated in tension device 12, integrated in compression member 14, and/or may include a discrete component relative to tension device 12 and compression member 14. It is understood that the above are only examples of attachment device 36 and should not be construed as limitations of attachment device 36 and that other attachment features may be possible.

Tension device 12 may include a fixture that may have at least one articulable joint, e.g., that may be pivotally attached to a plurality of sections. The fixture may include one or more segments or sections that may be held together by at least one joint.

The fixture may provide for controlled positioning of compression member 14. The fixture may also provide for controllable pressure application by compression member 14 to an anatomical location.

The fixture may be made from various materials, such as plastic, carbon fiber or a metal or metal alloy. Examples of suitable metals may include but not limited to, titanium, titanium-nickel alloys (such as Nitinol), tantalum, stainless steel, cobalt-chromium, aluminum, and the like. It is understood that the above metals are only examples and not to be construed as limitations. It is also understood that plastic, carbon fiber or metals are only examples and not be construed as limitation and that the fixture may be constructed from other material.

The sections of the fixture may be adjustably secured to each other in order to prevent and/or reduce movement, once secured, during a medical operation and in order to continually apply pressure via compression member 14. The length of the one or more sections may be adjustable or fixed. Adjustment of the length of one or more of the sections may be achieved by telescopically increasing or decreasing the length of a section, may be achieved by adding or replacing some of the sections, and/or by other suitable adjustment. The sections may have any suitable cross-sectional shape, for example, square or round in shape. The thickness of the sections may be varied, for example, depending on the amount of desired pressure for a specific procedure. The sections may be hollow or solid. It is understood that the above are only examples and should not be construed as limitations, other section dimensions may be used.

One or more joints may act as junctions by which the sections of tension device 12 may be adjustably secured to each other. In various embodiments, joints between sections of the tension device 12 may be hinging, gliding, pivoting, or the like. Joints may be secured in place to resist movement between the individual sections, for example using a locking mechanism that may comprise threading, latching or clamping mechanisms. The locking may be done manually, electronically, electromechanically, hydraulically, etc. Additionally, the individual sections of tension device 12 may be capable of being oriented at different angles relative to each other. Further, the individual sections of tension device 12 may be adjusted to be on different planes relative to each other, e.g., to provide for customized elevations and/or orientations relative to the anatomical location.

The number of sections may vary depending on location of the work area where tension device 12 may be attached, e.g., relative to the anatomical location at which compression member 14 may be positioned. The number of sections may also vary depending on the size of the patient, desired orientation of compression member, and the like. For example, a child patient may require a smaller number of sections and/or shorter sections as compared to an older or larger patient, who may require an increased number of sections and/or longer sections to achieve a desired positioning of compression member 14. Various additional and/or alternative considerations and user preferences may influence the number and/or length of the sections of tension device 12. An example of an apparatus that may suitable be employed as tension device 12 may include a BOOKWALTER™ retractor system made by Codman Inc., which is a division of Johnson and Johnson Inc. It is understood that the above example of tension device 12 is only for illustrative purposes and that other fixtures may be used.

As generally discussed above, the work area may be any location that may provide means for tension device 12 to be secured. A work area may include, for example, a railing on a bed, a table, a floor, a ceiling, a movable object or even a patient. However, it will be appreciated that other work areas may be utilized to secure tension device 12.

Contact surface 16 may include a portion of compression member 14 that may provide contact with an anatomical location. Contact surface 16 may, for example, include a portion of an individual prong or a ring. In some embodiments, contact surface 16 may be configured to be located proximate the anatomical location. Contact surface 16 may be curved, angled, straight, and/or of other suitable configuration.

As also generally described above, the anatomical location may include, but not limited to, an area around the common femoral artery, an area surrounding an entry point of a surgical device or a location requiring application of pressure during a surgical procedure.

The foregoing examples of contact surface 16 of a compression member 14 of compression apparatus 10 and the foregoing examples of anatomical locations in which compression apparatus 10 may be utilized are provided for the purpose of example only, and should not be construed as a limitation of the present disclosure. Compression apparatus 10 may be equally utilized for the application of continuous pressure to various other anatomical locations.

In an example embodiment, compression apparatus 10 may be to placed relative to a desired anatomical location with desired pressure applied to the anatomical location and/or anatomical structures. With the desired pressure applied via compression member 14 (e.g., and contact surface 16), tension device 12 may be secured (e.g., by tightening one or more locking devices associated with one or more joints of tension device 12) in order to maintain compression apparatus 10 in a desired position. In some embodiments, a relatively small adjustment in the pressure applied via compression member 14 and/or a location of compression member 14 may be made by loosening and/or adjusting one or more of the joints of tension device 12, telescopically adjusting compression member 14 (e.g., which may include one or more prongs, which may be telescopically adjusted as a group and/or individually) to thereby alter the applied pressure. In an embodiment, one or more of the prongs of compression member 14 may be telescopically adjusted using a hydraulic mechanism, or other suitable telescoping mechanism.

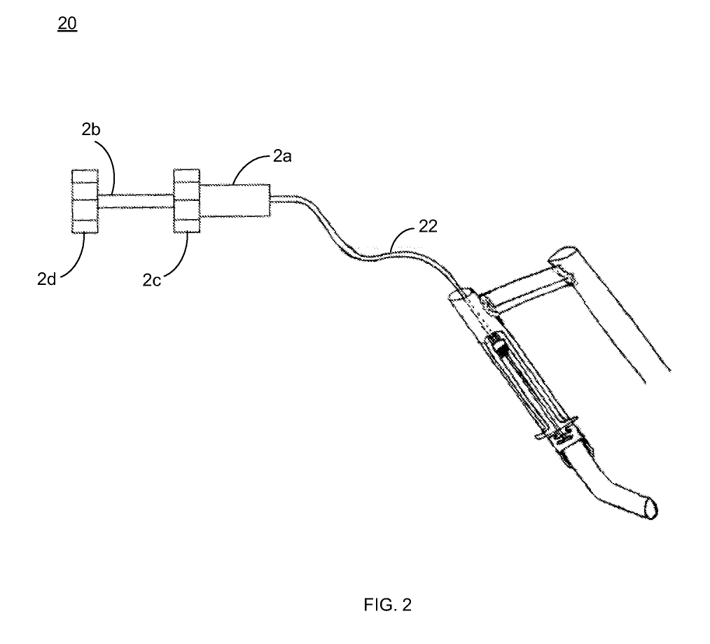

Referring to FIG. 2, there is shown hydraulic system 20 that may interact with compression apparatus 10, for example to allow one or more prongs of compression member 14 to be telescopically adjusted, e.g., to vary the pressure applied by one or more of the prongs of compression member 14. Consistent with the illustrated embodiment, hydraulic system 20 may include an application side that is proximal to compression member 14, a user side that is proximal to the operator controlling the applied pressure. In some embodiments, the application side and the user side may each include a hydraulic piston arrangement, e.g., which may allow pressure applied at the user side hydraulic piston to be transferred to the application side hydraulic piston. In an example embodiment, one or both of the application side hydraulic piston and the user side hydraulic piston may include a syringe. The application side syringe and the user side syringe may be fluidly coupled by tubing 22. Further, hydraulic system 20 may utilize a relatively non-compressible fluid as a hydraulic medium for transmitting pressure between the user side syringe and the application side syringe. Various suitable syringes and tubing 22 may be utilized. For example, the syringes may include relatively high pressure syringes, such as high density polyethylene syringes, and tube 22 may include high pressure tubing. Examples of syringes and tubing may be available from Merit Medical Systems, Inc.

Further, in some embodiments, one or more mechanism may be utilized to multiply to pressure applied at the user side to apply a greater pressure at the application side of hydraulic system 20. For example, a relatively smaller cross-sectional hydraulic piston at the user side and a relatively larger cross-sectional piston at the application side may allow a relatively smaller force applied to the user side hydraulic piston to apply a relatively larger force at the application side hydraulic piston. Additionally/alternatively, force multiplication may be achieved using a threaded ram 2b that may act against the user side syringe (e.g., which may be contained in support portion 2a). Consistent with such an embodiment, an operator may exert a force on the user side syringe by advancing threaded ram 2b (e.g., by turning knob 2d) against the user side syringe. Threaded ram 2b may threadably engage a cooperating thread in end piece 2c associated with support portion 2a. The threaded engagement between threaded ram 2b and cooperating threads in end piece 2c may provide a mechanical advantage that my act as a force multiplier, e.g., allowing the user to exert a relatively small force (e.g., associated with turning knob 2d) that may translate into a relatively large force at the application side syringe. In an example embodiment, the user side assembly (e.g., including the user side syringe, and assembly 2a-2d) may include a commercial vertebroplasty injector, such as a vertebroplasty injector made by Parallax Medical Inc. It is understood that the above user side injector is only an example and other alternatives for applying and controlling pressure may be used. In some embodiments, hydraulic system 20 may allow pressure applied using the hydraulic system 20 may be precisely controlled, e.g., which may prevent and/or minimize tissue harm or necrosis that may result from the application of excessive pressure.

It is understood that the length and diameter of the part 2a and part 2b may vary depending on various sizes of syringes to be used. For example, having a bigger syringe may allow for longer movement of one or more of the prongs or rings and hence would require a longer part 2a and part 2b.

Pressure tubing 22 that connects the application side to the user side may have different length depending on the location of the operator with regards to the anatomical location where compression member 14 may be used.

The application side hydraulic piston (e.g., which may include a syringe) may be incorporated within compression member 14 to allow the compression pressure to be applied by one or more of the prongs or rings as a result of a user applying pressure at the user side of hydraulic system 20. As can be seen in FIG. 2, the application side may be incorporated in at least one prong of compression member 14 to allow for telescopically extending the prong towards, and/or retract the prong away from, the anatomical location.

The pressure may be controlled using an output device that may display the applied pressure. While hydraulic system 20 has been described above, this is for the purpose of illustration, and should not be construed as a limitation of the present disclosure. Various additional/alternative hydraulic systems may equally be utilized.

As discussed above, compression member 14 may include a plurality of prongs. Prongs may be projections, fingers or rings. For illustration purposes, hereinafter, “prongs” will refer to finger shaped prongs as generally depicted in FIG. 1 for example. Prongs may include contact surfaces 16, which may provide the contact to anatomical location.

The plurality of prongs may provide localized compression to an anatomical location. The distance between one or more of the prongs may be adjustable and/or fixed relatively to at least another of the prongs. Additionally, the area inside a ring may also be adjustable or fixed. In some embodiments, the distance between the prongs, or the space on the inside of the rings, may be configured to allow, for example, access by an ultrasound probe, to allow a surgeon sufficient room to access a femoral artery, or the like. It is understood that the above are only examples and not to be considered as limitations. Other conditions may require more or less spacing between the prongs or inside the rings.

As discussed above, one or more of the prongs may be movable or fixed relative to one or more of the other prongs. For example, one or more of the prongs may be movable relative to connection feature 30 individually and/or in a group, which may allow a user to adjust the relative position and/or spacing of the prongs as required by the procedure, and/or aspects of the procedure being performed. As also generally discussed, the prongs may be also fixed together through connection feature 30 without the option or being movable. Instead, compression member 14 may be replaced with a compression member 14 having a configuration that may be suitable for the specific procedure being performed. In some embodiments, the prongs may be locked in place as they compress the tissues around the sheath using tension device 12. It is understood that the above are only examples of prongs configured relative to connection feature 30 and contact member 32 and not to be considered as limitations. Other configuration may be possible.

The prongs may be constructed having various different shapes to allow customization for specific procedures. The cross-sectional shape of the prongs may be cylindrical, rectangular, flat, etc. The prongs may include linear projections that may include curved and/or arcuate portions or geometries. The prongs may include one or more curved and/or angled segments adjacent a distal end of the prongs. The prongs may be made of various materials, such as plastic (e.g., thus disposable), carbon fiber (e.g., thus be radiolucent), a metallic material (e.g., thus durable and sterilizable).

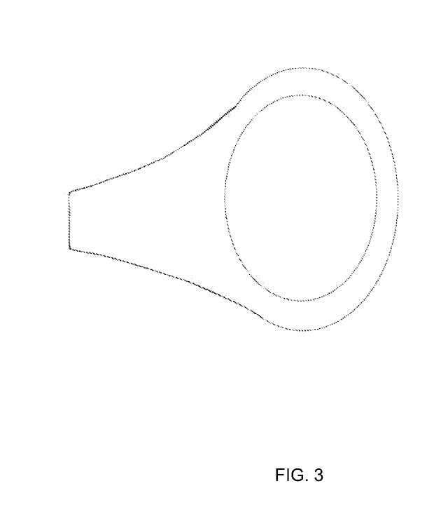

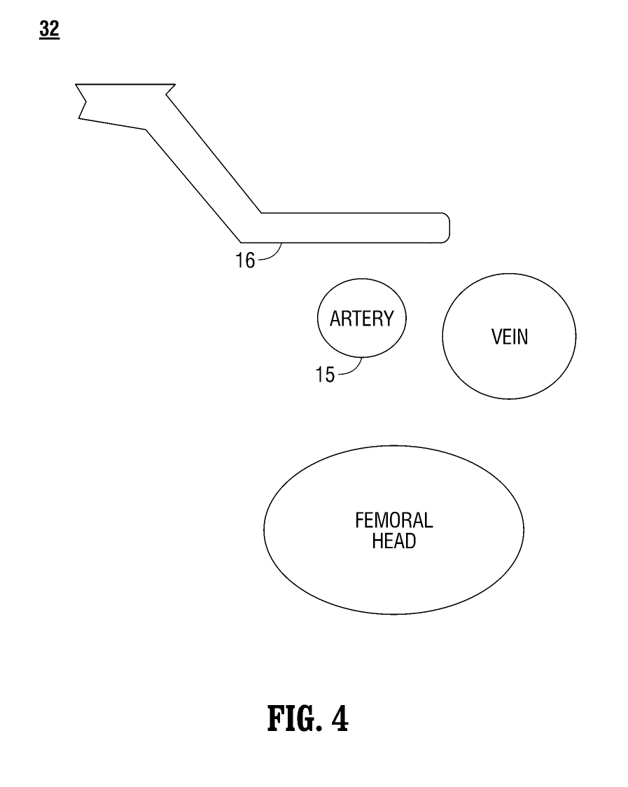

Compression member 14 may include at least a ring. Referring to FIGS. 3 and 4, there is shown, one or more rings, e.g., which may be included on compression member 14 instead, and/or in addition to, the one or more prongs. Rings may be used in similar procedures and/or in a generally similar manner as the prongs described herein-above. For example, one or more rings may be used in connection with various types of arterial access based procedures, e.g., that may require the common femoral artery, or other vascular lumen, as an access point. Referring to FIG. 4, there is shown contact member 32 that may include rings or prongs having contact surface 16. Rings or prongs may provide pressure at an anatomical location, for example above an artery, vein and/or a femoral artery, which may allow a physician to perform more complex procedures requiring larger sheath sizes via percutaneous technique. Rings may be used for example to encircle a vascular sheath. It is understood that the above is an example and not to be a limitation of the disclosure and other usage for the ring may be possible.

Rings may be made from similar material as prongs. For example, rings may be made from a plastic material, a carbon fiber material, a metallic material, as well as various other suitable materials.

The one or more rings may include, for example, circular rings, oval-shaped rings, rectangular-shaped rings, as well as rings having various other geometries. In case of a circular ring, the diameter may be selected depending on the anatomical location and depending on the patient. For example if the patient is a child or a smaller person, a relatively smaller diameter may be utilized. However, if the patient is an adult or a larger patient, a relatively larger diameter may be utilized. The diameter of a ring may be fixed or may be dynamically modified during usage. For example, rings having fixed diameters may be used and replaced as desired. Further, a ring diameter may be modified by elongating the circumference through a telescoping motion for example. This can be achieved by a user, electronically, and/or mechanically utilizing a mechanism that may allow movement of at least one side of the ring to telescopically extend or contract. It is understood that the above are examples and not to be limitations of the disclosure and other configurations may be possible.

The thickness of the rings may be selected depending on the patient (child versus adult or small versus large), and/or user need and/or preference. It is understood that the above are examples and not to be limitations of the disclosure other factors may govern the requirement for thicker or thinner rings.

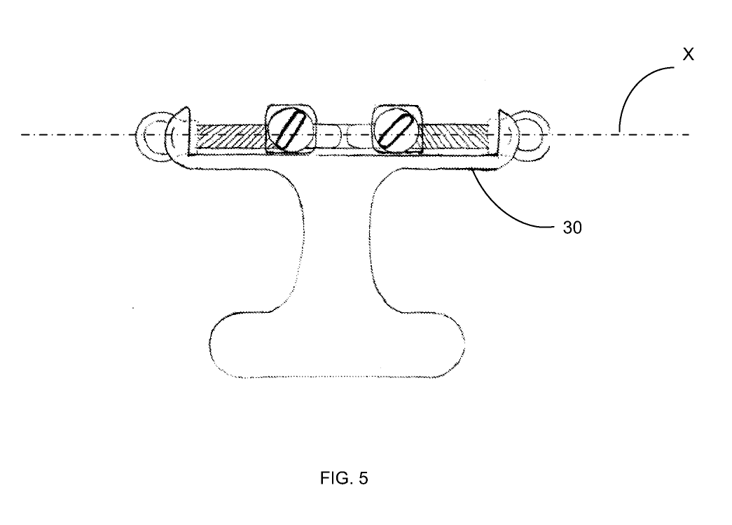

In some embodiments, compression member 14 may include at least two outer prongs and two inner prongs. Referring to FIGS. 5-8, in various embodiments the prongs may be provided having different sizes. For example, in some embodiments the two outer prongs may be thicker compared to the inner prongs. Further, the distances between the prongs may be adjusted and/or provided to allow for smaller space between adjacent prongs. It is understood that the above are examples and not to be limitations of the disclosure and that other sizes and separation may be achieved.

In some embodiments, the two outer prongs may be stationary and the two inner prongs may be individually adjustable. Referring to FIG. 5, the adjustable prongs may move around an axis of connection feature 30 in order to allow the operator more room to access the anatomical location. For example, prongs may adjustably move along an axis (i.e. axis X) of connection feature 30 in order to alter the spacing between two or more of the prongs relative to one another. The lateral adjustment along the axis (i.e. axis X) of connection feature 30 may be achieved hydraulically, using a threaded adjustment mechanism, electronically, and/or using various additional/alternative mechanisms. The outward lateral movement may allow the prongs to increase separation between two or more prongs, and thus may apply pressure to different regions of an anatomical site. In some embodiments, an inward lateral movement may allow the prongs to come closer together, e.g., which may in some embodiments allow the soft tissues over an incision or puncture to be pinched, e.g., in order to stop and/or control the bleeding. In some embodiments, when a desired location of the prongs is achieved, the prongs may be locked in place through a locking mechanism. Examples of locking mechanism may be achieved through threading, latching or clamping. It is understood that the above are only examples and not to be limitations of the disclosure and that other adjustments to the prongs may be possible.

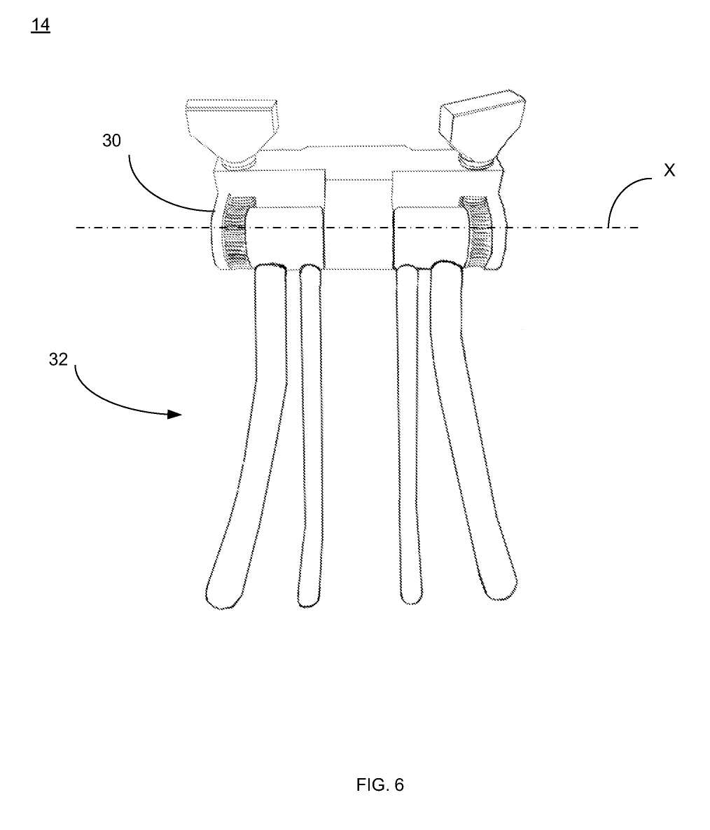

Referring to FIG. 6, there is shown, compression member 14 which may include contact member 32 and connection feature 30. The prongs within compression member 14 may be adjusted rotationally around axis X of connection feature 30. Prongs may be rotated individually or in a group. For example, in FIG. 6, a group of two prongs may be rotated around axis X of connection feature 30 in order to achieve a desired compression across an anatomical location. Further, the group of two prongs may be rotated away relative to the anatomical location in order to provide the operator with enhanced access to the anatomical location. The above is used as an example for illustrative reason and other grouping or prongs or rotational configuration may be achieved.

The prongs may be secured in place by locking them at a desired location. Examples of locking mechanism may be achieved through threading, latching or clamping. It is understood that the above are only examples and not to be limitations of the disclosure and that other ways of locking prongs in position may be possible.

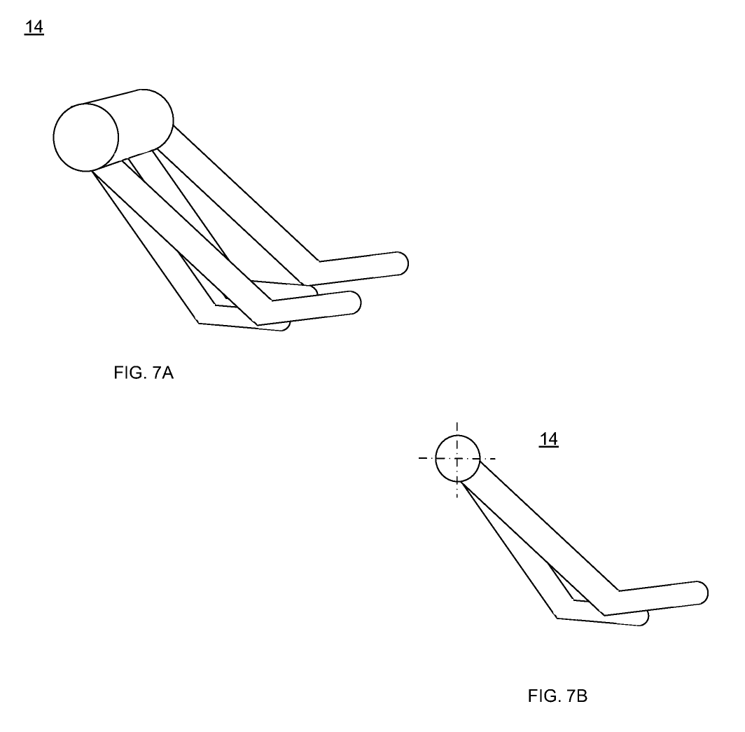

The at least two prongs may be staggered. Referring to FIG. 7A and for illustrative purposes only, assuming compression member 14 includes four prongs, the two inner prongs may be positioned lower than the two outer prongs for possibly providing more compression using the two inner prongs. The two outer prongs may hold the skin away from the anatomical area to prevent excess skin from covering the anatomical area where the operator may be planning to make the incision. FIG. 7B shows a cross-sectional view of compression member 14 that include 4 prongs in a staggered configuration. In other embodiments, the prongs in compression member 14 are positioned on the same place relative to each other. It is understood that the above are only examples and not to be limitations of the disclosure and that other staggering configurations may be possible.

The amount of staggering may be adjustable depending on the patient or the procedure performed. The amount of staggering may be manually adjusted or may be achieved by replacing compression member 14 with a more staggered setup. Alternatively, all the prongs may be on a same plane to allow for increased pressure on the outer prongs. It is understood that the above are only examples and not to be limitations of the disclosure and that other adjustments to the prongs may be possible.

At least a portion of the plurality of prongs may be telescopically adjustable.

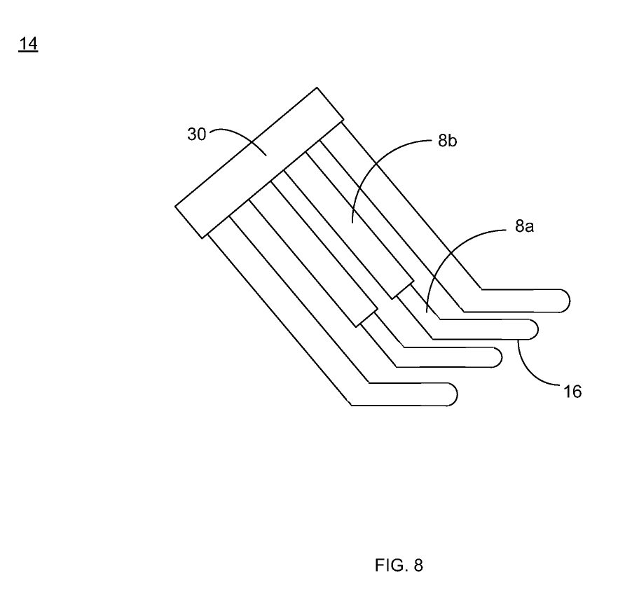

Referring to FIG. 8, prongs may be configured to have separate parts that move one within another in order to achieve a variety of lengths. Telescopically adjusting the prongs may allow the operator to adjust the amount of pressure to be applied to an anatomical location. Movement of parts in a telescoping way may be achieved through sliding of parts within each other or threading of one part into the other part. For example, prongs could have two parts one part 8a (distal to contact surface 16) and another part 8b (proximal to contact surface 16). Part 8a may be configured to move within part 8b or part 8b maybe configured to move within part 8a. The movement may be achieved through sliding, threading or hydraulically. When a desired extension is achieved through telescopically moving parts 8a and 8b, these parts may be held in place by a locking mechanism. Examples of locking mechanism may be achieved through threading, latching or clamping. It is understood that the above are only examples and not to be limitations of the disclosure and that other telescopically adjusted prongs may be possible.

Compression apparatus 10 may include at least one articulable joint 20 attached between compression member 14 and tension device 12. At least one joint may be positioned between compression member 14 and tension device in order to freely move around different axes until compression member 14 may be locked in place. This may allow the operator to position compression member 14 at a desired location by rotating compression member 14 relative to tension device 12. The joint may be incorporated directly into contact member 32 (which may include compression member 14 and connection feature 30) or may be attached to tension device 12. The joint may be secured in place by a locking mechanism that might comprise threading, latching or clamping. It is understood that the above are only examples of a locking mechanism and not to be limitations of the disclosure and that other locking mechanisms may be possible.

In other embodiments, it might be desirable to implement multiple compression apparatuses 10 during a medical procedure. For example, two compression apparatuses 10 may be employed during Endovascular Aortic/Aneurysm Repair (EVAR) and Thoracic Endovascular Aortic/Aneurysm Repair (TEVAR) for treating Abdominal Aortic Aneurysms (AAA) and Thoracic Aortic Aneurysms (TAA). The first compression apparatus 10 may be attached through one tension device 12 to a work area and the second compression apparatus may be attached to a second tension device to the same work area or to a different work area. It is understood that the above are only examples of setting up multiple compression apparatuses 10 and should not be construed as limitations, other configurations may be possible.

What is claimed is:

1. A compression apparatus comprising:
a tension device configured to be secured relative to a work area; and
a compression member adjustably coupled to the tension device and including a contact surface configured to apply pressure relative to an anatomical location.

2. The apparatus of claim 1, wherein the compression member includes a plurality of prongs

3. The apparatus of claim 1, wherein the compression member includes at least two prongs.

4. The apparatus of claim 1, wherein the compression member includes at least a ring.

5. The apparatus of claim 1, wherein the compression member includes at least two outer prongs and two inner prongs.

6. The apparatus of claim 2, wherein at least a portion of the plurality of prongs are telescopically adjustable.

7. The apparatus of claim 3, wherein the at least two prongs are staggered.

8. The apparatus of claim 3, wherein the at least two prongs are on a same plane.

9. The apparatus of claim 1, wherein the compression member further includes an articulable joint feature proximal the tension device.

10. The apparatus of claim 1, wherein the tension device includes a fixture having at least one articulable joint pivotally attached to a plurality of sections.

11. A compression apparatus comprising:
a tension device secured relative to a work area;
a compression member adjustably coupled to the tension device, and including a contact surface configured to apply pressure relative to an anatomical location; and
at least one articulable joint attached between the compression member and the tension device.

12. The compression apparatus of claim 11, wherein the compression member includes a plurality of prongs.

13. The compression apparatus of claim 11, wherein the compression member includes a least a ring.

14. The compression apparatus of claim 11, wherein the at least one articulable joint is at least partially disposed between at least two prongs.

15. The compression apparatus of claim 12, wherein the at least a portion of the plurality of prongs are individually adjustable.

16. The compression apparatus of claim 11, wherein the tension device includes a Bookwalter retractor.

17. The compression apparatus of claim 14, wherein the at least two prongs are staggered.

18. The compression apparatus of claim 14, wherein the at least two prongs are on a same plane.

19. The compression apparatus of claim 14, wherein the at least two prongs are individually adjustable.

20. The compression apparatus of claim 14, wherein the at least two prongs include two outer prongs and two inner prongs.

21. The compression apparatus of claim 20, wherein the two outer prongs are stationary and the two inner prongs are individually adjustable.

22. A compression apparatus comprising:
a connection feature;
at least one contact member associated with the connection feature; and
an attachment device associated with the connection feature for positioning of the at least one contact member relative to an anatomical location.

23. The compression apparatus of claim 22, wherein the connection feature is a crosspiece.

24. The compression apparatus of claim 23, wherein the contact member includes at least two prongs.

25. The compression apparatus of claim 23, wherein the contact member is at least a ring.

26. The compression apparatus of claim 25, wherein the at least two prongs are fixedly attached to the connection feature.

27. The compression apparatus of claim 25, wherein the at least two prongs are individually adjustable.

28. The compression apparatus of claim 25, wherein the at least two prongs include two outer prongs and two inner prongs.

29. The compression apparatus of claim 28, wherein the two outer prongs are stationary and the two inner prongs are individually adjustable.
